(19) United States
(12) Patent Application Publication
(10) Pub. No.: US 2022/0047826 A1
(43) Pub. Date: Feb. 17, 2022

(54) PORTABLE VENTILATION DEVICE

(71) Applicant: JK2L CO., LTD., Kaohsiung City (TW)

(72) Inventor: JIN-SHU WU, Kaohsiung City (TW)

(21) Appl. No.: 16/991,081

(22) Filed: Aug. 12, 2020

(51) Int. Cl. A61M 16/00; A61M 16/10; A41D 13/11; A61M 16/20

(52) U.S. Cl. CPC A61M 16/0066; A61M 16/107; A61M 2209/088; A61M 16/208; A61M 2205/8206; A41D 13/11

(57) ABSTRACT

A portable ventilation device is detachably attached to a face mask and communicated with a covered space formed between the face mask and a user wearing the face mask. The portable ventilation device includes a cover and a fan assembly. The cover has an attachment structure that allows the cover to be detachably attached to the face mask. The cover has an air inlet portion and an air outlet portion communicated with the air inlet portion. The fan assembly is positioned between the air inlet portion and the air outlet portion, and serves to introduce an external airflow into the covered space inside the face mask. The external airflow enters the cover through the air inlet portion and enters the covered space through the air outlet portion, thereby facilitating ventilation in the face mask and improving comfort of wearing the face mask.

### BACKGROUND OF THE INVENTION

### 1. Field of the Invention

The present invention relates to a ventilation device, and more particularly to a portable ventilation device that is designed to be detachably attached to a face mask.

### 2. Description of the Related Art

In the highly industrialized world with heavy traffic, waste gas deriving from manufacturing and transportation have led to an increasingly polluted environment. When breathing in air with dust and particulate matter (PM) in a long-term manner, people can be at risk suffering from health hazards like irritation, asthma, cardiovascular diseases and respiratory diseases. Thus, people often wear face masks when going out to protect themselves from harmful impurities and droplet transmission diseases.

The face mask products currently sold on the market are generally composed of a mask body and two ear loops connected to two lateral edges of the mask body. Therein, the mask body covers the oronasal area of a wearer to prevent direct exposure to the exterior. In addition, the mask body is made of filter materials, so when an external airflow enters it, airborne dust and impurities can be substantively caught by the mask body, leaving the filtered air clean and fresh.

However, long-term use of face masks can make users have muggy and uncomfortable feeling, which decreases their willingness to wear face masks.

### SUMMARY OF THE INVENTION

For addressing the problems of the traditional face masks about muggy feeling and uncomfortable wearing experience, the present invention provides a portable ventilation device, which is portable and convenient to use. The disclosed device can be easily detachably attached to a general face mask, or used with a check valve to complement a respirator to send filtered air into the face mask, thereby providing a wearer of the mask with fresh air and facilitating ventilation inside the face mask. In this way, the disclosed device optimizes humidity and apparent temperature in the covered space, thereby making the face mask more comfortable to wear.

For achieving the foregoing objective, the present invention provides a portable ventilation device, being detachably attached to a face mask, and communicated with a covered space formed between the face mask and a user wearing the face mask, the portable ventilation device comprising: a cover, having an attachment structure by means of which the cover is detachably attached to the face mask, an air inlet portion, and an air outlet portion communicated with the air inlet portion; and a fan assembly, being deposited on the cover and comprising a battery unit and an air-drawing unit, the battery unit powering the air-drawing unit, the air-drawing unit being arranged between the air inlet portion and the air outlet portion, and the air-drawing unit introducing an external airflow into the covered space of the face mask so that the external airflow enters the cover through the air inlet portion and then enters the covered space through the air outlet portion.

Thereby, the disclosed device is portable. When a user can detachably attach the cover to a face mask, the device introduces an external airflow into the covered space of the face mask to enhance ventilation inside the face mask and optimize the humidity and apparent temperature in the covered space, thereby making the face mask comfortable to wear.

### BRIEF DESCRIPTION OF THE DRAWINGS

FIG. 1 is a perspective view of a first embodiment of the present invention.

FIG. 2 is a partially exploded view of the first embodiment of the present invention.

FIG. 3 is an applied view of the first embodiment of the present invention.

FIG. 4 is an applied view showing a face mask with the device of the first embodiment of the present invention worn by a user.

FIG. 5 is a cross-sectional applied view of the first embodiment of the present invention.

FIG. 6 is an applied view of the second embodiment of the present invention.

FIG. 7 is a cross-sectional applied view of the second embodiment of the present invention.

FIG. 8 is an applied view of a third embodiment of the present invention.

FIG. 9 is a cross-sectional applied view of the third embodiment of the present invention.

FIG. 10 is a perspective view of a fourth embodiment of the present invention.

FIG. 11 is a bottom perspective view of the fourth embodiment of the present invention.

FIG. 12 is an applied view of the fourth embodiment of the present invention.

FIG. 13 is a cross-sectional applied view of the fourth embodiment of the present invention.

FIG. 14 is an applied view of a fifth embodiment of the present invention.

FIG. 15 is a lateral applied view of the fifth embodiment of the present invention.

FIG. 16 is a cross-sectional applied view of the fifth embodiment of the present invention.

### DETAILED DESCRIPTION OF THE INVENTION

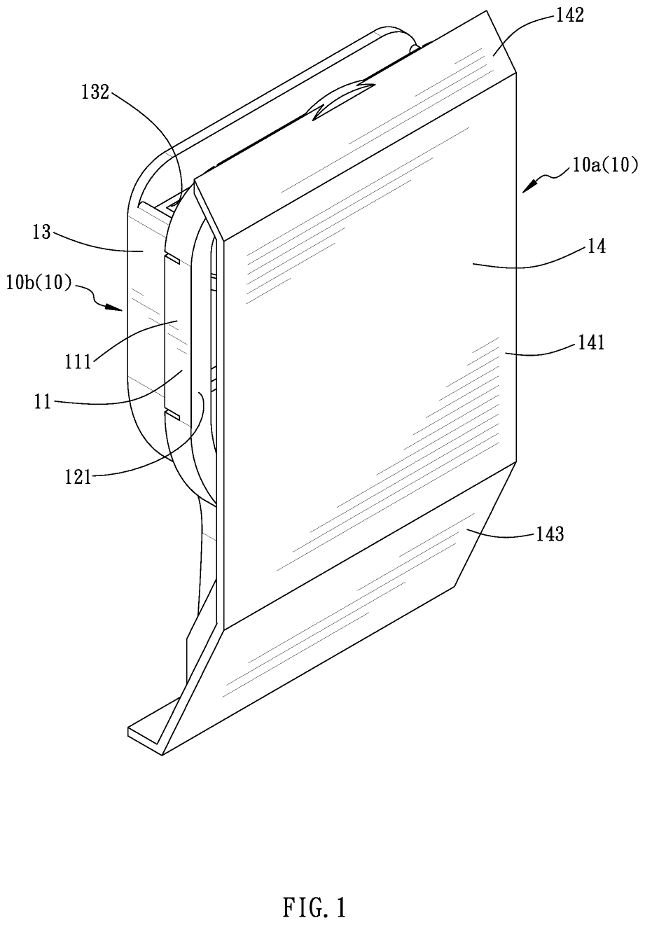

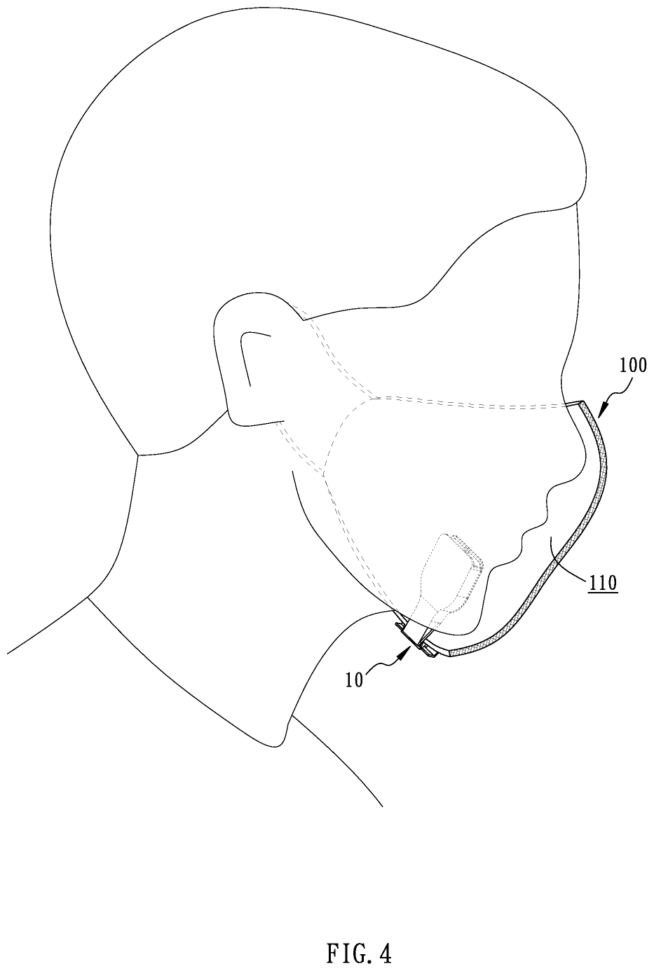

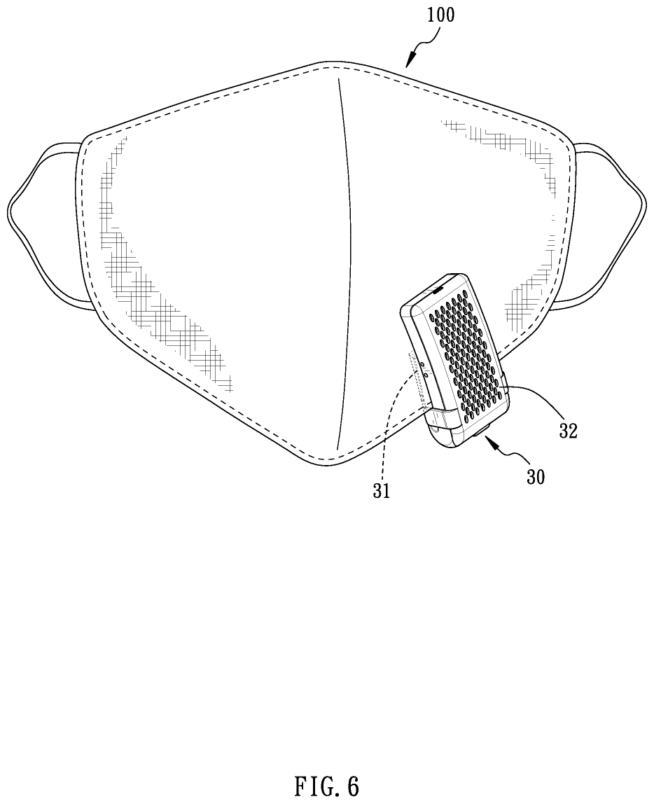

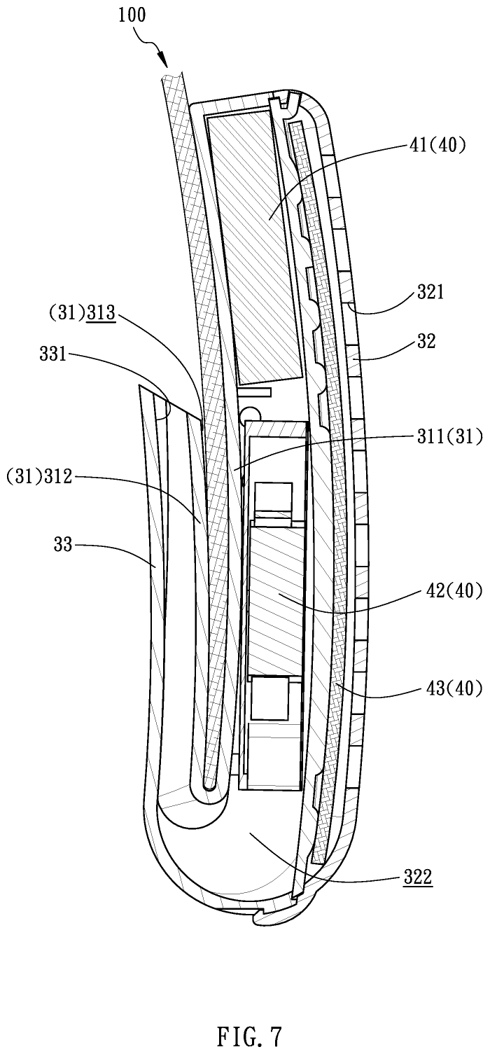

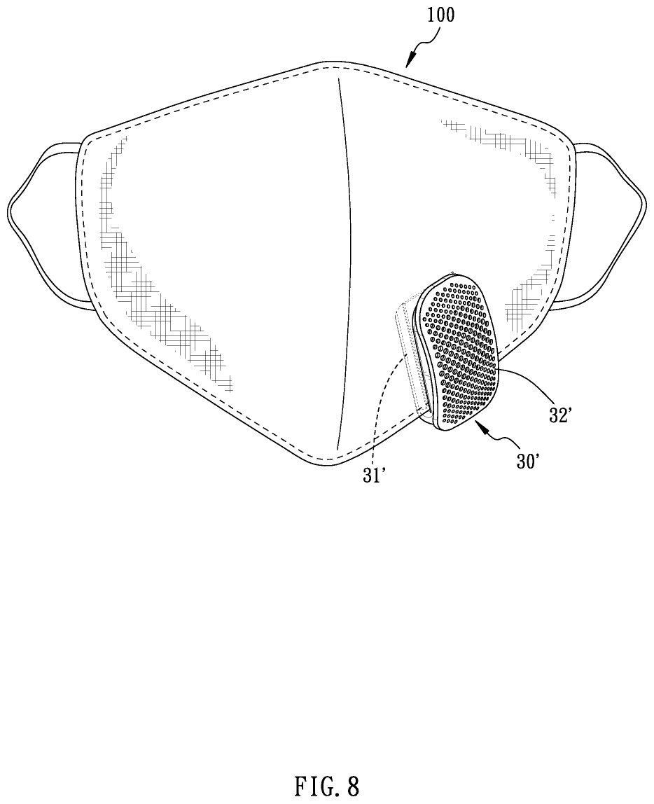

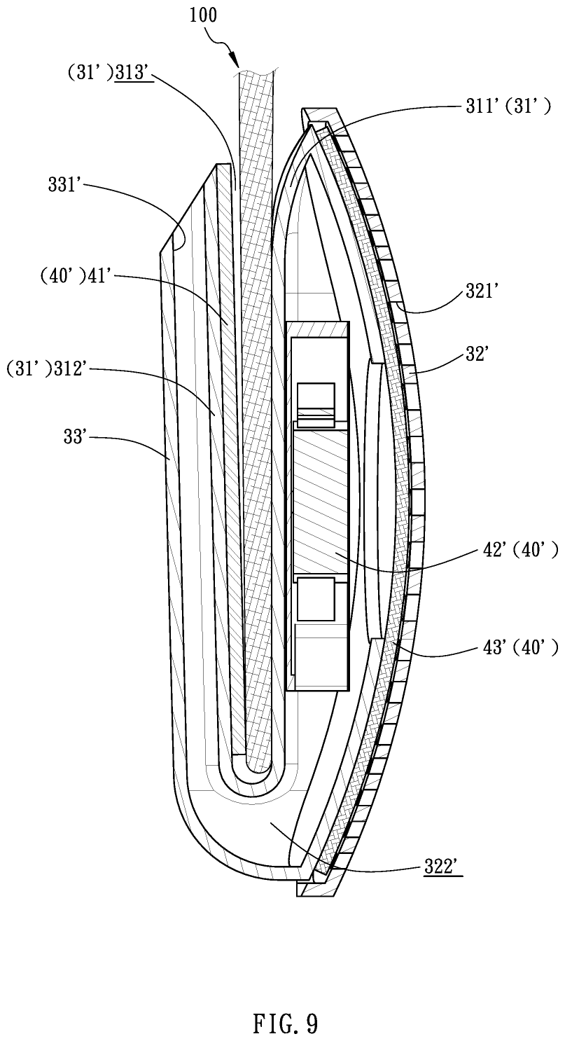

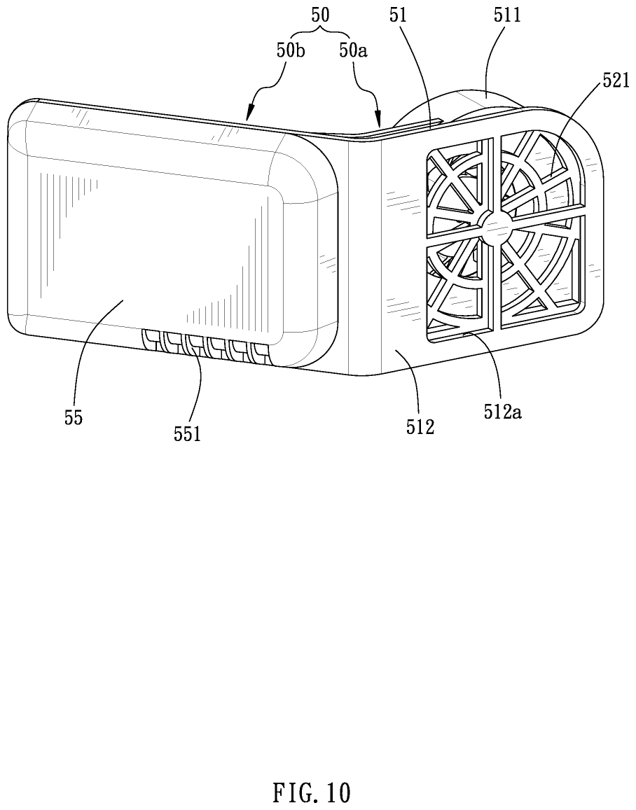

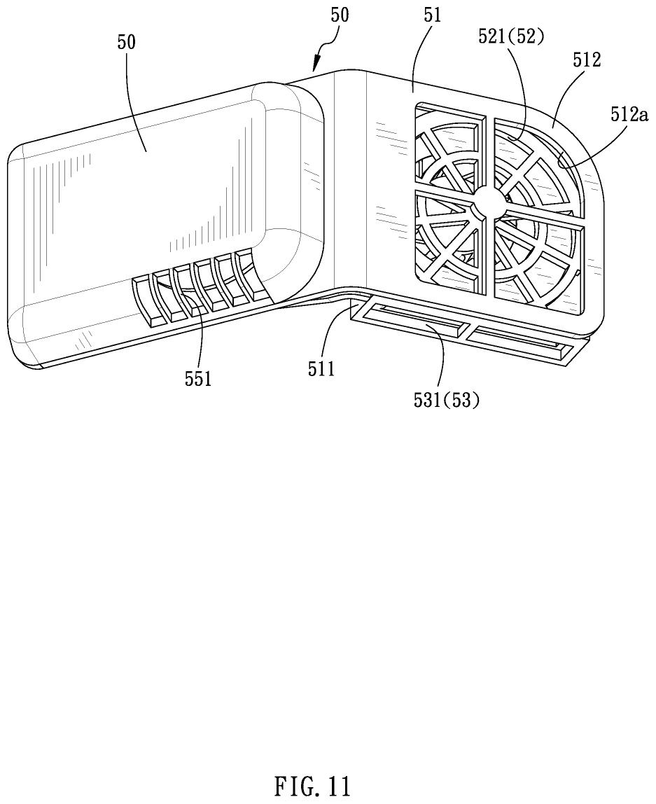

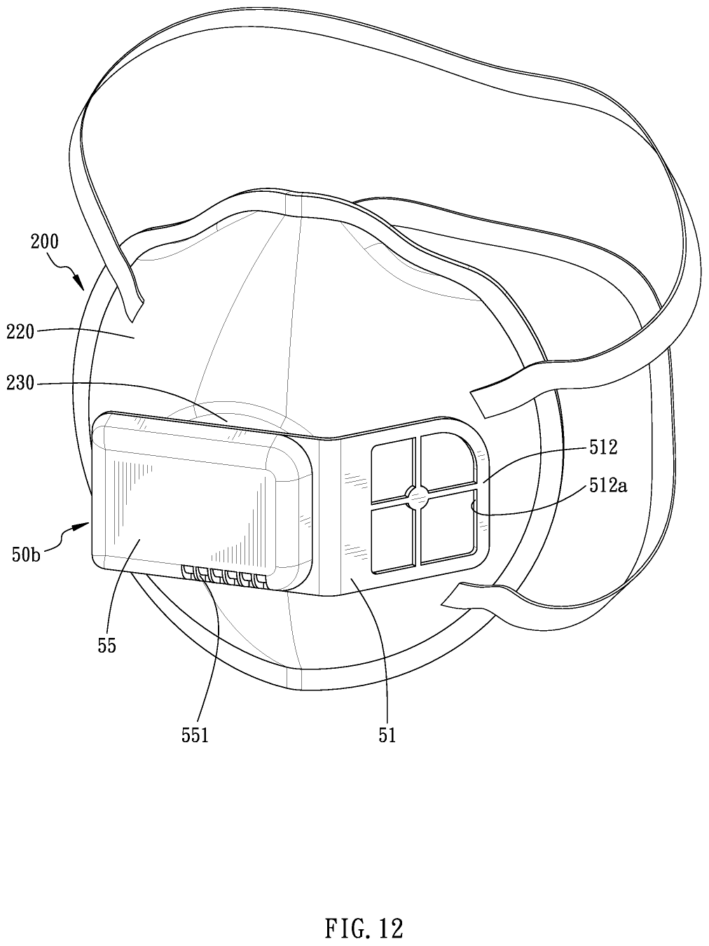

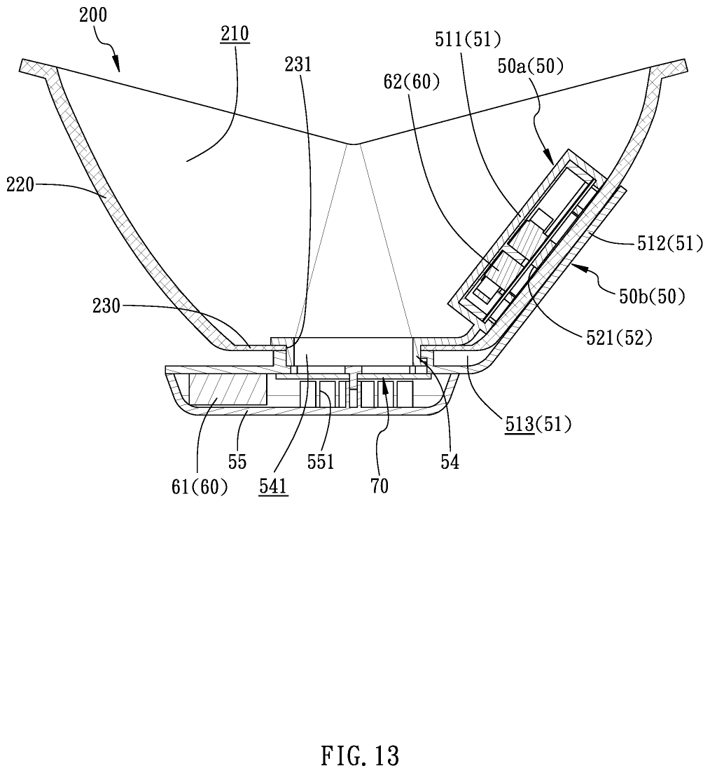

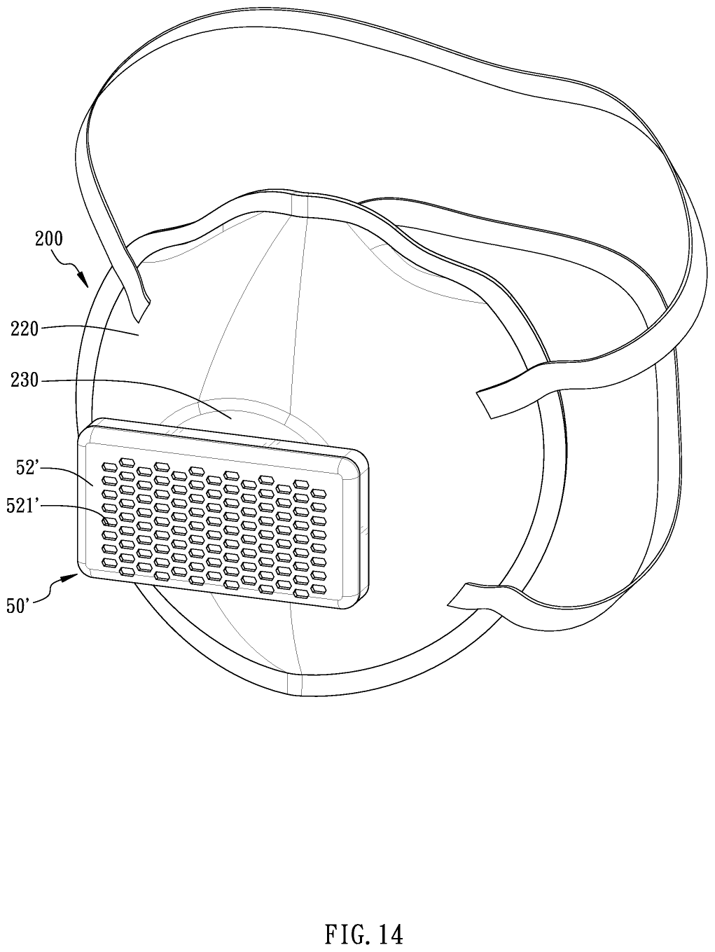

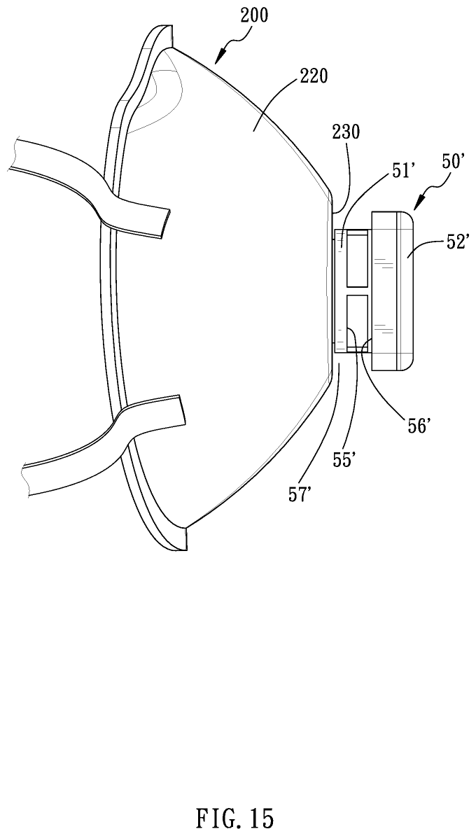

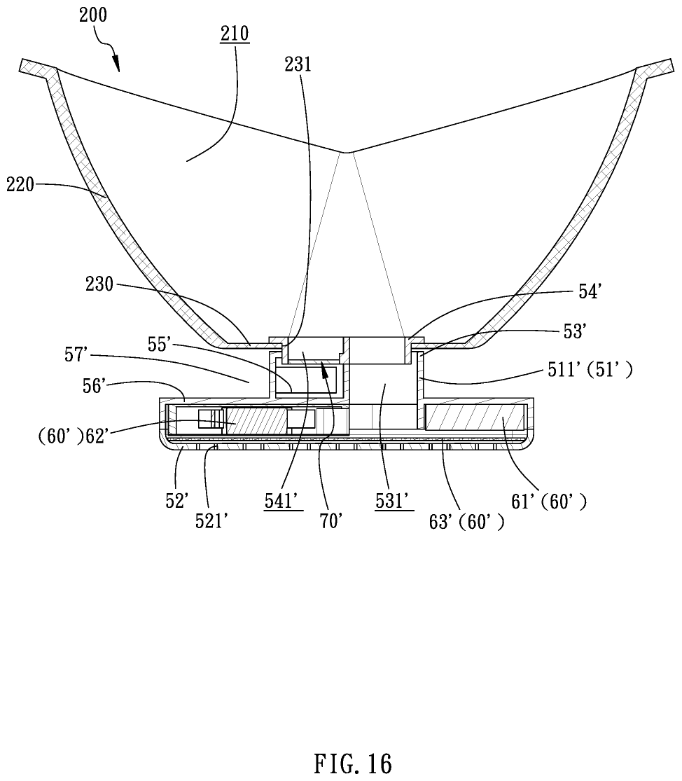

The invention as well as a preferred mode of use, further objectives and advantages thereof will be best understood by reference to the following detailed description of illustrative embodiments when read in conjunction with the accompanying drawings FIG. 1 through FIG. 16. FIG. 1 through FIG. 5 depict a first embodiment of the present invention. FIG. 6 and FIG. 7 depict a second embodiment of the present invention. FIG. 8 and FIG. 9 depict a third embodiment of the present invention. FIG. 10 through FIG. 13 depict a fourth embodiment of the present invention. FIG. 14 through FIG. 16 depict a fifth embodiment of the present invention.

The disclosed portable ventilation device is designed to be detachably attached to a face mask 100. Therein, the face mask 100 may be a commercially available one, like a cotton face mask, an activated-carbon face mask, a non-woven face mask or an N95 respirator, without limitation. When used by a user, the face mask 100 defines a covered space 110 between its inner surface and the face of the user.

Referring to FIG. 1 through FIG. 5, in the first embodiment of the present invention, the portable ventilation device comprises a cover 10 and a fan assembly 20.

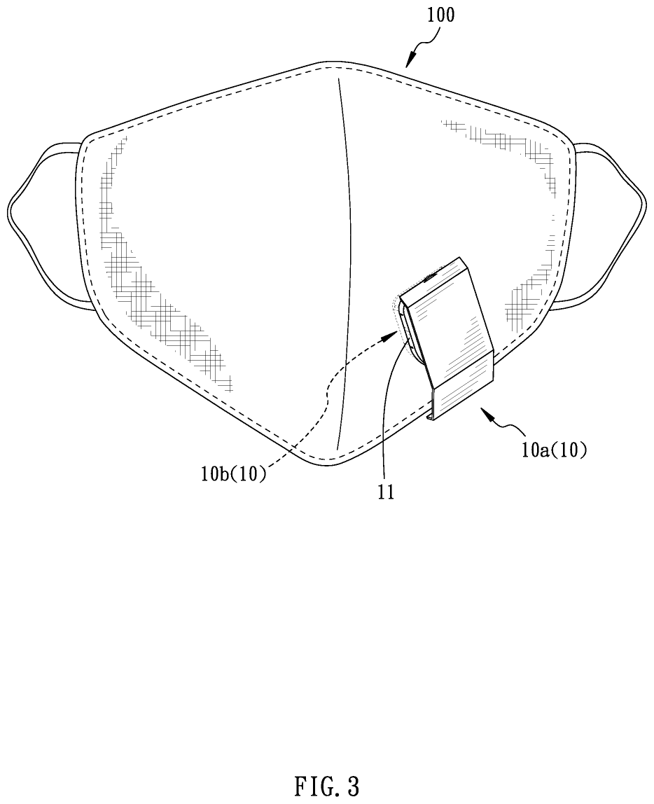

The cover 10 has a flat, roughly rectangular shape, and is specifically sized not to hinder the sight of the user who is using it. While the illustrated cover 10 is designed to be directly assembled to the lower edge of the face mask 100, it may be alternatively attached to either of the lateral edges of the face mask 100. Also referring to FIG. 3 through FIG. 5, the cover 10 has an attachment structure 11. The cover 10 is designed to be detachably attached on to the face mask 100 by means of the attachment structure 11. The cover 10 has an air inlet portion 12 and an air outlet portion 13 that is communicated with the air inlet portion 12. In a preferred embodiment, the attachment structure 11 is positioned between the air inlet portion 12 and the air outlet portion 13, and is engaged with the peripheral edge of the face mask 100.

More specifically, the cover 10 comprises a first cover 10a and a second cover 10b. The first cover 10a has the air inlet portion 12, and the second cover 10b has the air outlet portion 13. The attachment structure 11 has a frame 111 and a guide plate 112. The frame 111 is located on the first cover 10a and connected to the air inlet portion 12. The guide plate 112 is located on the second cover 10b and connected to the air outlet portion 13. The guide plate 112 is peripherally provided with a flange 112a. The frame 111 is detachably attached to the flange 112a of the guide plate 112, so that the first cover 10a is positioned at the outside of the face mask 100 while the second cover 10b is positioned at the inside of the face mask 100, with the face mask 100 sandwiched and fixed between the frame 111 and the guide plate 112 of the attachment structure 11.

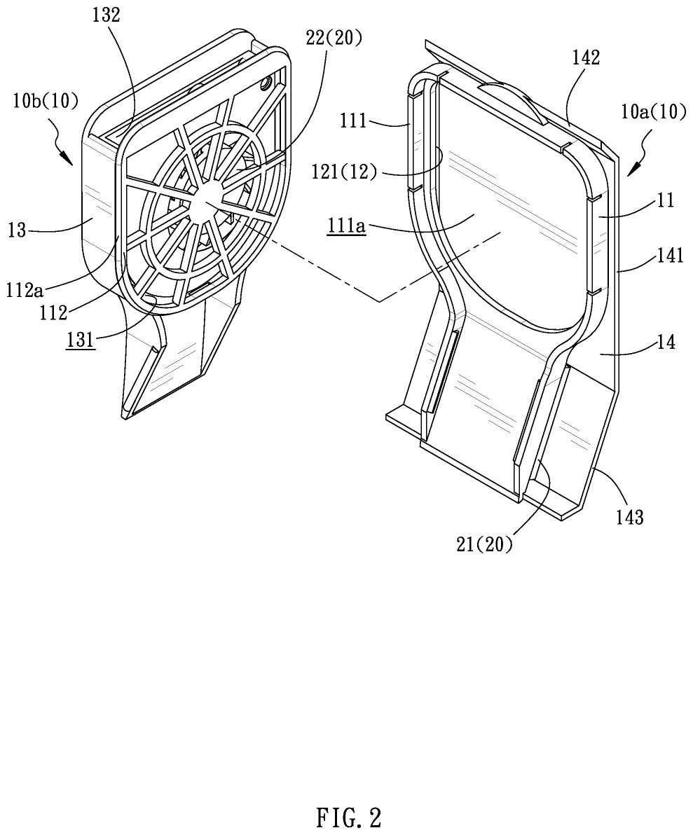

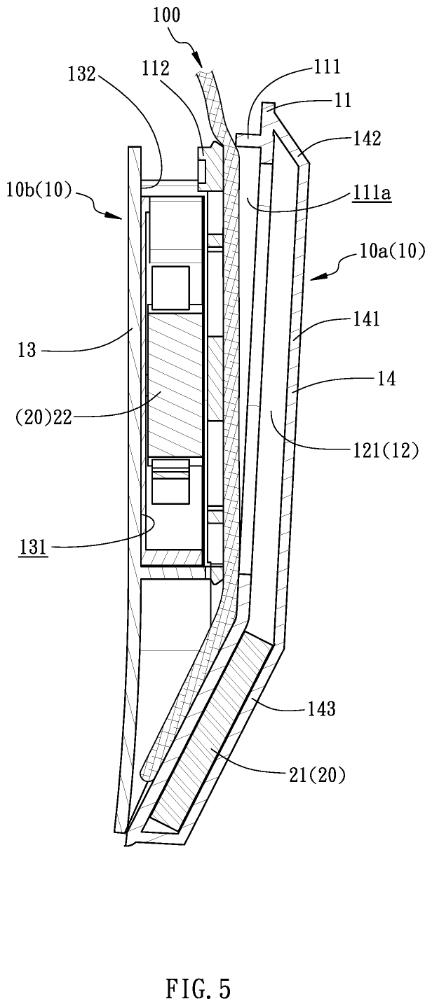

Please further refer to FIG. 2 and FIG. 5. The first cover 10a comprises a panel 14 connected to the frame 111. The panel 14 has a flat segment 141, a first inclined segment 142, and a second inclined segment 143. The first inclined segment 142 and the second inclined segment 143 extend inclinedly away from two ends of the flat segment 141, respectively. Two ends of the frame 111 are connected to the first inclined segment 142 and the second inclined segment 143, respectively. The air inlet portion 12 has a lateral slot 121 formed between the frame 111 and the flat segment 141, and the frame 111 has therein a hollow 111a that is aligned with the flat segment 141 and communicated with the lateral slot 121 of the air inlet portion 12.

In a preferred embodiment, the air outlet portion 13 contains therein an accommodating space 131. The accommodating space 131 is communicated with the hollow 111a of the frame 111 through the guide plate 112. The air outlet portion 13 has its one edge provided with an air outlet 132 that is communicated with the accommodating space 131. The air outlet 132 extends in a direction that is perpendicular to the axis of the hollow 111a.

The fan assembly 20 is installed in the cover 10 and comprises a battery unit 21 and an air-drawing unit 22. The battery unit 21 serves to power the air-drawing unit 22. The air-drawing unit 22 introduces an external airflow to the covered space 110 of the face mask 100. The external airflow enters the cover 10 through the air inlet portion 12, and enters the covered space 110 through the air outlet 132 of the air outlet portion 13. In a preferred embodiment, the battery unit 21 is a rechargeable battery, located between the frame 111 of the first cover 10a and the second inclined segment 143 of the panel 14. The air-drawing unit 22 may be a micro fan or a blower, installed in the accommodating space 131 of the second cover 10b and located between the air inlet portion and the air outlet portion. The air-drawing unit 22 may be turned on and off using a power switch.

In the present embodiment, the face mask 100 acts as a filter that filters the introduced external airflow. When air outside the face mask 100 is introduced into the air inlet portion 12 by the air-drawing unit 22, the face mask 100 filters out and blocks dust and microbes carried by the external airflow so that the air enters the covered space 110 of the face mask 100 through the air outlet 132 of the air outlet portion 13 is clean and fresh for the user to consume.

Moreover, by introducing the filtered air to the covered space 110 of the face mask 100, the fan assembly 20 improves ventilation inside the face mask 100, and in turn optimizes the humidity and apparent temperature in the covered space 110, to make wearing the face mask 100 in a hot environment a comfortable experience.

Referring to FIG. 6 and FIG. 7, in the second embodiment of the present invention, a portable ventilation device comprises a cover 30 and a fan assembly 40 similar to their counterparts as described previously. The second embodiment is different from the first embodiment in additional variation of the cover 30. Thus, the cover 30 of the second embodiment is different from the cover 10 of the first embodiment in terms of design and form.

Specifically, the cover 30 comprises an attachment structure 31, and air inlet portion 32, and an air outlet portion 33 similar to their counterparts as described previously. Therein, the attachment structure 31 has a first jaw portion 311, a second jaw portion 312, and a gap 313 formed between the first jaw portion 311 and the second jaw portion 312. In the present embodiment, the first jaw portion 311 has a length greater than the length of the second jaw portion 312, and the gap 313 fittingly receives the face mask 100, so that the first jaw portion 311 and the second jaw portion 312 jointly hold the face mask 100 from inside and outside and prevent the cover 30 from coming off the face mask 100, thereby achieve firm and efficient attachment.

Referring to FIG. 7, in a preferred embodiment, the air inlet portion 32 is connected to the first jaw portion 311 and located outside the face mask 100. The air inlet portion 32 has a plurality of vents 321 distributed across its side away from the first jaw portion 311. The air inlet portion 32 contains therein an accommodating space 322 communicated with the vents 321. The air outlet portion 33 is connected to the second jaw portion 312. The air outlet portion 33 has an air outlet 331 communicated with the accommodating space 322. The air outlet 331 extends in a direction that is parallel to the direction in which the gap 313 receives the face mask and the air outlet 331 is communicated with the covered space 110 of the face mask 100.

The fan assembly 40 comprises a battery unit 41, and air-drawing unit 42, and a filter unit 43 similar to their counterparts as described previously. In the present embodiment, the battery unit 41, the air-drawing unit 42, and the filter unit 43 are installed in the accommodating space 322 of the air inlet portion 32. Therein, the battery unit 41 is electrically connected to the air-drawing unit 42. The filter unit 43 is installed between the vents 321 and the air-drawing unit 42 for filtering the external airflow introduced into the air inlet portion 32, so that the filtered air can be guided to the air outlet 331 of the air outlet portion 33 for discharge. In this way, when the fan assembly 40 is turned on to draw external air for filtration, the incoming air enters the cover 30 through the vents 321 of the air inlet portion 32. At this time, the filter unit 43 filters out dust and PM, so that the filtered air can be guided to the air outlet 331 of the air outlet portion 33 and then introduced into the low covered space 110 of the face mask 100, thereby providing the user with fresher air.

Referring to FIG. 8 and FIG. 9, in the third embodiment of the present invention, a portable ventilation device comprises a cover 30′ and a fan assembly 40′ similar to their counterparts as described previously. The third embodiment is different from the second embodiment in that the first jaw portion 311′ of the cover 30′ has a length that is equal to the length of the second jaw portion 312′. Therein, the battery unit 41′ of the fan assembly 40′ is installed in the gap 313′ of the attachment structure 31′ and electrically connected to the air-drawing unit 42′. The air-drawing unit 42′ and the filter unit 43′ of the fan assembly 40′ are installed in the accommodating space 322′ of the air inlet portion 32′. This arrangement helps to minimize the length of the cover 30′ and in turn the overall volume of the device, making the device more portable and convenient to use.

Now please refer to FIG. 10 through FIG. 13 for the fourth embodiment of the present invention. The portable ventilation device herein comprises a cover 50 and a fan assembly 60 similar to their counterparts as described previously. The fourth embodiment is different from the first embodiment in the mode of the face mask 200 and the additional structural variation of the cover 50. Thus, the cover 50 of the fourth embodiment is different from the cover 10 of the first embodiment in terms of design and form.

Specifically, the face mask 200 in the present embodiment is an N95 respirator. The face mask 200 defines a covered space 210 at its inside. Therein, the face mask 200 has a bowl-shaped mask body 220, which defines therein a covered space 210. The face mask 200 has a mount portion 230 whose part positionally corresponding to the oronasal area of a user wearing it is flush with one end of the mask body 220. The mount portion 230 is formed with an opening 231.

The cover 50 has an attachment structure 51, an air inlet portion 52, an air outlet portion 53 all similar to their counterparts as described previously, and an air-discharging portion 54. The air-discharging portion 54 at its part positionally corresponding to the oronasal area of a user wearing it abuts against the mount portion 230 while being fit in the opening 231. More specifically, the cover 50 of the present embodiment comprises a first cover 50a and a second cover 50b. The first cover 50a has its one end provided with the air-discharging portion 54. The air-discharging portion 54 has a discharging channel 541 communicated with the covered space 210. The second cover 50b has its one end provided with a fastening portion 55. The fastening portion 55 when detachably attached to the air-discharging portion 54 abuts against the outside of the mount portion 230 of the face mask 200. The fastening portion 55 has an air-discharging hole 551 communicated with the discharging channel 541.

In addition, the attachment structure 51 has a first jaw portion 511 and a second jaw portion 512. The first jaw portion 511 is away from the air-discharging portion 54 and is located on the opposite end of the first cover 10a. The second jaw portion 512 is away from the fastening portion 55 and is located on the opposite end of the second cover 50b. Thereby, a gap 513 is formed between the first jaw portion 511 and the second jaw portion 512. In the present embodiment, when the periphery of the mask body 220 of the face mask 200 is fit into the a gap 513 of the attachment structure 51, the first jaw portion 511 is located in the covered space 210 of the face mask 200, and the second jaw portion 512 is located at the outside of the face mask 200. More specifically, the air inlet portion 52 and the air outlet portion 53 are deposited on the first jaw portion 511. Therein, the air inlet portion 52 abuts against the inside of the face mask 200 and has an air hole 521 communicated with the gap 513.

The second jaw portion 512 has an air-guiding port 512a aligned with the air hole 521, so that the gap 513 is communicated with the air-guiding port 512a and the air hole 521. The air outlet portion 53 has an air outlet 531, which extends in a direction that is perpendicular to the direction in which the air hole 521 extends.

In a preferred embodiment, the disclosed device further comprises a one-way discharging valve 70, which is located between the air-discharging portion 54 and the fastening portion 55. When a user wearing the mask with the disclosed device exhales, an exhaled airflow is generated in the covered space 210 of the face mask 200. At this time, most of the exhaled airflow is gathered and guided to the discharging channel 541 of the cover 10 through the opening 231 of the face mask 200, so that the exhaled airflow passes through the discharging channel 541 and pushes the one-way discharging valve 70 to open, thereby allowing the exhaled airflow to be discharged through the air-discharging hole 551.

On the contrary, when the user wearing the face mask 200 breathes in, the one-way discharging valve 70 is closed. The user can turn on the air-drawing unit 62 of the fan assembly 60 to draw in air outside the face mask 200. At this time, the mask body 220 of the face mask 200 filters out and blocks most dust and microbes in the introduced air, which is then guided to the air outlet 531 through the air hole 521 of the air inlet portion 52 and sent into the covered space 210 of the face mask 200. Thereby, the user can consume the filtered, fresh air directly in the covered space 210 of the face mask 200. Additionally, the air-drawing unit 62 helps to cool down the air temperature in the covered space 210 by improving ventilation in the face mask 200, thereby making the face mask 200 more comfortable to wear.

Referring also to FIG. 14 through FIG. 16, in the fifth embodiment of the present invention, a portable ventilation device comprises a cover 50′, a fan assembly 60′, and a one-way discharging valve 70′ similar to their counterparts as described previously. The fourth embodiment is different from the first embodiment in that the cover 50′ has its air inlet portion 52′ and air outlet portion 53′ located at the outside of the face mask 200. Specifically, the attachment structure 51′ of the cover 50′ has a fastening portion 511′. The fastening portion 511′ is detachably mounted around the air-discharging portion 54′, so that the air inlet portion 52′ and the air outlet portion 53′ are located at the outside of the face mask 200. Therein, the air inlet portion 52′ has a plurality of air holes 521′ formed at one side of the cover 50′ and the air outlet portion 53′ has an air-discharging channel 531′ communicated with the air holes 521′. The air-discharging channel 531′ has an axial direction parallel to the direction in which the air holes 521′ extend. More specifically, the fan assembly 60′ further comprises a filter unit 63′. The filter unit 63′ is located between the air holes 521′ of the air inlet portion 52′ and the air-drawing unit 62′, for filtering the external airflow introduced into the air inlet portion 52′. The filtered air is then guided to the air outlet portion 53′ for discharging.

In addition, the cover has an air-discharging hole 55′ that is communicated with the discharging channel 541′ of the air-discharging portion 54′. The air-discharging hole 55′ extends in a direction that is perpendicular to an axis of the discharging channel 541′. In a preferred embodiment, the cover 50′ further has a retaining portion 56′. The retaining portion 56′ extends outward form the fastening portion 511′, so that an air-discharging area 57′ is formed between the retaining portion 56′ and the fastening portion 511′ and is communicated with the air-discharging hole 55′. It is to be noted that since the air-discharging hole 55′ is arranged at the lateral edge of the fastening portion 511′, and the air-discharging hole 55′ extends in a direction that is perpendicular to the axis of the discharging channel 541′, so that the air-discharging hole 55′ forms a lateral air-discharging port, and then since the retaining portion 56′ of the cover 50′ can prevents the air from flowing along the axis of the discharging channel 541′, the exhaled air can be gathered and guide to the air-discharging area 57′ through the air-discharging hole 55′ for discharging.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A portable ventilation device, being detachably attached to a face mask, and communicated with a covered space formed between the face mask and a user wearing the face mask, the portable ventilation device comprising:
a cover, having an attachment structure by means of which the cover is detachably attached to the face mask, an air inlet portion, and an air outlet portion communicated with the air inlet portion; and
a fan assembly, being deposited on the cover and comprising a battery unit and an air-drawing unit, the battery unit powering the air-drawing unit, the air-drawing unit being arranged between the air inlet portion and the air outlet portion, and the air-drawing unit introducing an external airflow into the covered space of the face mask so that the external airflow enters the cover through the air inlet portion and then enters the covered space through the air outlet portion.

2. The portable ventilation device of claim 1, wherein the attachment structure is located between the air inlet portion and the air outlet portion, and is fastened to a peripheral edge of the face mask.

3. The portable ventilation device of claim 2, wherein the cover comprises a first cover and a second cover, in which the first cover has the air inlet portion and the second cover has the air outlet portion, while the attachment structure has a frame and a guide plate, the frame being deposited on the first cover and connected to the air inlet portion, the guide plate being deposited on the second cover and connected to the air outlet portion, and the frame being detachably attached to a periphery of the guide plate, so that the first cover is located at the outside of the face mask, and the second cover is located at the inside of the face mask, while the face mask is sandwiched and fixed between the frame and the guide plate.

4. The portable ventilation device of claim 3, wherein the first cover comprises a panel connected to the frame, in which the panel has a flat segment, a first inclined segment, and a second inclined segment, the first inclined segment and the second inclined segment inclinedly extending outward from two ends of the flat segment, respectively, the frame having two ends thereof connected to the first inclined segment and the second inclined segment, respectively, the air inlet portion having a lateral slot formed between the frame and the flat segment, and the frame defining therein a hollow aligned with the flat segment.

5. The portable ventilation device of claim 4, wherein the air outlet portion defines therein an accommodating space that is communicated with the hollow of the frame through the guide plate, and the air-drawing unit is installed in the accommodating space, in which the air outlet portion has an air outlet communicated with the accommodating space and the air outlet extends in a direction that is perpendicular to an axial direction of the hollow.

6. The portable ventilation device of claim 2, wherein the attachment structure has a first jaw portion, a second jaw portion, and a gap formed between the first jaw portion and the second jaw portion, in which the first jaw portion has a length greater than or equal to a length of the second jaw portion, and the gap fittingly receives the face mask, so that the first jaw portion and the second jaw portion jointly hold the face mask from inside and outside.

7. The portable ventilation device of claim 6, wherein the air inlet portion is connected to the first jaw portion and has a plurality of air holes at one side thereof away from the first jaw portion, in which the air inlet portion defines therein an accommodating space communicated with the air holes, and wherein the air outlet portion is connected to the second jaw portion and the air outlet portion has an air outlet communicated with the accommodating space, in which the air outlet extends in a direction that is parallel to a direction in which the gap receives the face mask and faces the covered space of the face mask.

8. The portable ventilation device of claim 7, wherein the fan assembly further comprises a filter unit that is deposited between the air holes and the air-drawing unit for filtering the external airflow entering the air inlet portion and the filtered external airflow is guided to the air outlet portion for discharging.

9. The portable ventilation device of claim 1, wherein the face mask has a bowl-shaped mask body defining therein the covered space, and the face mask has a mount portion whose part positionally corresponding to an oronasal area of the user is flush with one end of the mask body, in which the mount portion is provided with an opening, and the cover further has an air-discharging portion whose part positionally corresponding to the oronasal area of the user abuts against the mount portion and receives in the opening.

10. The portable ventilation device of claim 9, wherein the cover comprises a first cover and a second cover, in which the first cover has one end thereof provided with the air-discharging portion that has a discharging channel communicated with the covered space, and the second cover has one end thereof provided with a fastening portion that is detachably attached to the air-discharging portion and abuts against the outside of the mount portion, wherein the fastening portion has an air-discharging hole communicated with the discharging channel.

11. The portable ventilation device of claim 10, further comprises a one-way discharging valve that is deposited between the air-discharging portion and the fastening portion, so that an exhaled airflow generated in the covered space of the face mask when a user wearing the face mask exhales passes through the discharging channel and pushes the one-way discharging valve to open, thereby allowing the exhaled airflow to be discharged through the air-discharging hole.

12. The portable ventilation device of claim 10, wherein the attachment structure has a first jaw portion and a second jaw portion, in which the first jaw portion is away from the air-discharging portion and is located on an opposite end of the first cover, while the second jaw portion is away from the fastening portion and is located on an opposite end of the second cover, so that a gap is formed between the first jaw portion and the second jaw portion, and when the gap of the attachment structure fittingly receives a periphery of the mount portion, the first jaw portion is positioned in the covered space of the face mask and the second jaw portion is positioned at the outside of the face mask.

13. The portable ventilation device of claim 12, wherein the air inlet portion and the air outlet portion are deposited on the first jaw portion, in which the air inlet portion abuts against the inside of the face mask and has an air hole communicated with the gap, while the second jaw portion has an air-guiding port aligned with the air hole, so that the gap is communicated with the air-guiding port and the air hole, and in which the air outlet portion has an air outlet that extends in a direction perpendicular to a direction in which the vent extends.

14. The portable ventilation device of claim 9, wherein the attachment structure has a fastening portion that is detachably mounted around the air-discharging portion, so that the air inlet portion and the air outlet portion are located at the outside the face mask, in which the air inlet portion has a plurality of air holes provided at one side of the cover, and the air outlet portion has an air-discharging channel communicated with the air holes, with the air-discharging channel having an axial direction parallel to a direction in which the air holes extend, and wherein the fan assembly further comprises a filter unit that is located between the air holes and the air-drawing unit and serves to filter the external airflow entering the air inlet so that the filtered external airflow is then guided to the air outlet portion for discharging.

15. The portable ventilation device of claim 14, further comprising a one-way discharging valve that is deposited on the air-discharging portion, wherein the air-discharging portion has a discharging channel communicated with the covered space, and the one-way discharging valve normally covers the discharging channel, in which the cover has an air-discharging hole that extends in a direction perpendicular to an axial direction of the discharging channel, so that an exhaled airflow generated in the covered space of the face mask when a user wearing the face mask exhales passes through the discharging channel and pushes the one-way discharging valve to open, thereby allowing the exhaled airflow to be discharged through the air-discharging hole.
